(19) United States
(12) Patent Application Publication
(10) Pub. No.: US 2020/0268292 A1
(43) Pub. Date: Aug. 27, 2020

(54) pH SENSOR AND CALIBRATION METHOD FOR THE pH SENSOR

(71) Applicant: University of Southampton, Southampton, Hampshire (GB)

(72) Inventors: Hywel Morgan, Wiltshire (GB); Roel Mingels, Southampton (GB)

(21) Appl. No.: 16/646,489

(22) PCT Filed: Sep. 13, 2018
(86) PCT No.: PCT/GB2018/052612

§ 371 Date: Mar. 11, 2020

(30) Foreign Application Priority Data
Sep. 13, 2017 (GB) 1714735.6

(51) Int. Cl. A61B 5/145; A61B 5/00; A61B 5/1473

(52) U.S. Cl. CPC A61B 5/14539; G01N 27/4167; A61B 5/1473; A61B 5/6875

(57) ABSTRACT

A pH sensor for measuring pH levels within a measurement environment is described. The sensor comprises a reference electrode, a pH sensitive electrode, and a controller, for measuring the potential difference between the pH sensitive electrode and the reference electrode, the measured potential difference being indicative of a pH level at the pH sensitive electrode. The controller is operable to apply a voltage across first and second electrodes to control the pH level at the pH sensitive electrode, and to measure the potential difference between the pH sensitive electrode and the reference electrode following the application of the recalibration voltage. In this way, recalibration of the pH sensor is possible within the measurement environment.

### TECHNICAL FIELD

The present invention relates to a pH sensor and a calibration method for said sensor. Embodiments of the present invention may be implemented in an implantable or remotely located device. Embodiments of the present invention relate to a pH sensor (and optionally dissolved oxygen (DO) sensor) for long-term, real-time, measurement of physical parameters in a measurement system, and to a calibration method of calibrating the pH sensor (and optionally the dissolved oxygen sensor) in situ.

### BACKGROUND

In a previous patent application by the present Applicant, an intra-uterine monitoring system is described. The system comprises an implantable device housing different sensors, including sensors for measuring pH levels and DO (dissolved oxygen) concentration, to aid in the diagnosis of sub-fertility.

A problem with sensors in this form of application, is that they become less accurate over time. Reasons for this inaccuracy include sensor drift, biofouling, surface changes and reference electrode instability. To maintain optimal performance recalibration is required. This is very challenging to achieve in situ in a remote environment such as the human body, or environmental measurement. Miniature solid state pH sensors such as ISFETs (ion-sensitive field effect transistors) and metal-oxide based sensors (Iridium Oxide, Platinum Oxide, Ruthenium etc.) can be implemented in small, remote, and low power sensing devices. Prior to measurements being taken, calibrations are carried out in solutions of known pH to achieve optimal performance. In applications where access to the sensor is not possible or undesirable, for example in implantable applications, these calibrations outside of the measurement environment are not possible during the course of the measurement.

Previous proposals for addressing such limitations include improving reference electrode stability (but this does not account for changes on the sensing electrode surface), the use of reference sensors (such as REFETs), but this is difficult to achieve in practice and not compatible with non-ISFET sensors, the use of drift prediction and correction based on previously obtained data and recalibration using spectroscopic/optical techniques such as sensitive dyes in situ.

The present invention seeks to provide an alternative method to overcome these limitations.

In U.S. Pat. Nos. 4,961,163 and 5,046,028, principles for use in macro-scale and more specifically glass pH sensors is described. In situ control of local pH using Boron doped diamond ring disk electrode is described in T. L. Read, E. Bitziou, M. B. Joseph, J. V. Macpherson; 2013; Analytical Chemistry doi: 10.1021. In US20140008244, a principle of pH generation is described for bio-sensing applications. In US 20080202944, a principle of electrolysis is used to calibrate and condition dissolved oxygen sensors.

### SUMMARY OF THE INVENTION

According to a first aspect of the present invention, there is provided a pH sensor for measuring pH levels within a measurement environment, the sensor comprising:
a reference electrode;
a pH sensitive electrode,
a controller, for measuring the potential difference between the pH sensitive electrode and the reference electrode, the measured potential difference being indicative of a pH level at the pH sensitive electrode;
and
wherein the controller is operable:
   to apply a voltage across first and second electrodes to control the pH level at the pH sensitive electrode, and
   to measure the potential difference between the pH sensitive electrode and the reference electrode following the application of the voltage.

The first and second electrodes may be considered as a pair of electrolysis electrodes across which a predetermined (DC recalibration) voltage is applied to induce a flow of current which is sufficient to cause an electrolysis reaction in an aqueous solution within which the first and second electrodes are disposed. The first and second electrodes are an anode and cathode respectively, or a cathode and anode respectively, depending on the polarity of the applied voltage. It will be appreciated that the steps of applying the voltage across the first and second electrodes (to cause the electrolysis reaction) and measuring the resulting potential difference, may in this way be carried out while the pH sensor is in the measurement environment (rather than the pH sensor being required to be removed from the measurement environment for recalibration).

The controller (as part of the pH sensor (device) itself) may be operable to recalibrate the pH sensor based on the measurement. Alternatively, the recalibration may take place externally of the pH sensor itself. In other words, the pH sensor itself may simply be able to measure voltages and/or currents for output to an external device, with the external device interpreting the measured voltage and/or current as a particular pH level. In this case, the calibration of the pH sensor will be carried out by updating a mapping between measured voltage and pH at the external device, for example by modifying a look up table or one or more parameters of a mathematical expression.

The controller may be operable, while the voltage is being applied, to take a plurality of potential measurements at known times following the initial application of the DC recalibration voltage, and be operable subsequently to correlate the plurality of potential measurements with expected pH levels at the respective times. Alternatively, the controller is operable to apply the voltage with a first polarity until a first steady-state pH level is reached, and to take a first potential difference measurement at the first steady-state pH level, and then to apply the voltage with a second polarity opposite to the first polarity until a second steady-state pH level is reached, and to take a second potential difference measurement at the second steady-state pH level. In either case, the controller may be operable to determine a line of best fit through the correlated potential difference measurements and pH levels to obtain a gradient and/or zero crossing for a function used to determine pH levels from the measured voltage. It will therefore be appreciated that calibration values can be obtained in a variety of ways, such as by previous assessment of the measured pH at the start of the measurement in situ, using values of the pH in the case a solution of known pH is used, or derived from a steady state reached by application of the recalibration potential for a specific time period.

In its simplest form, the pH sensor can be any shape of planar electrode in the vicinity of a planar electrolysis electrode at a predetermined distance. The first electrolysis electrode may substantially surround (in a 2D plane) the pH sensitive electrode. The pH sensitive electrode may in particular comprise a substantially disk-shaped part and the first electrolysis electrode comprise a ring-shaped part which substantially surrounds the disk-shaped part of the pH sensitive electrode, or vice versa. Alternatively, to achieve a steady-state faster, the pH electrode may be a recessed disc, surrounded at the top by an electrolysis electrode, or vice versa.

All can be in a single large electrode lay-out or in smaller array type systems.

The DC recalibration voltage should exceed the electrolysis potential of water, approximately 1.23V between the two electrolysis electrodes, to initiate the reaction generating the pH change. The rate of pH change will be dependent on the current passed.

The pH sensor may be a glass pH probe, ISFET or any metal oxide based sensor, including Iridium Oxide (IrOx), which is a metal-oxide metal-oxide sensor. The present invention is particularly applicable to solid-state type sensors.

The measurement environment may be within a human or animal body, for example within a uterus. Alternatively, the measurement environment may be a remote industrial application, for example water-treatment systems, or a remote environmental application, for example deep sea monitoring.

According to another aspect of the invention, there is provided a multi-sensor device comprising a pH sensor according to the above and a dissolved oxygen sensor, the dissolved oxygen sensor comprising the first and second electrodes, one of the first and second electrodes being used as the working electrode of the dissolved oxygen sensor. Preferably, the other of the first and second electrodes is used as a counter electrode for the dissolved oxygen sensor.

The reference electrode of the pH sensor may be a common reference electrode for use with the dissolved oxygen sensor.

The controller may be operable to measure a dissolved oxygen level at the working electrode following the application of the DC calibration voltage to obtain a single point calibration of the dissolved oxygen sensor at a high dissolved oxygen concentration.

The pH sensitive electrode, the reference electrode and the first and second electrolysis electrodes may be disposed on a substrate. The substrate may be any type, for example silicon, glass, polyethylene or a printed circuit board. The pH sensitive electrode, the reference electrode and the first and second electrolysis electrodes may be disposed on the same side of the substrate. Alternatively, the pH sensitive electrode and the first electrolysis electrode may be disposed on a first side of the substrate and the reference electrode and the second electrolysis electrode may be disposed on a second, opposite, side of the substrate.

An electrolyte may be provided in a region bounded by the substrate and a semi-permeable membrane, the region containing at least the pH sensitive electrode and the first electrolysis electrode, diffusion of ions and oxygen from the measurement environment into the electrolyte occurring via the semi-permeable membrane.

According to another aspect of the invention, there is provided a method of calibrating a pH sensor within a measurement environment, the method comprising the steps of applying a voltage across first and second electrodes to influence the pH level at a pH sensitive electrode, and measuring the potential difference between the pH sensitive electrode and a reference electrode following the application of the voltage.

The method may comprise calibrating both the solid-state pH sensor and a dissolved oxygen sensor integrated with the pH sensor, and comprise a step of measuring a dissolved oxygen level at the first electrolysis electrode following the application of the DC calibration voltage to obtain a single point calibration of the dissolved oxygen sensor at a high dissolved oxygen concentration.

It will be appreciated therefore that the present techniques may provide a self-calibration method for miniaturized solid-state pH sensors. Many commercial applications may be found for this technique, including the monitoring of pH in in vivo applications (e.g. implantable devices), monitoring of pH in environmental applications (e.g. at sea), or bench-top solid-state pH meters.

More particularly, the present technique addresses the problem of sensor/reference electrode defects which occur during extended usage. By employing the self-calibration method described herein, the current calibration status of the sensor can be obtained. Major fields of application include: inaccessible locations such as in vivo environments and distant environmental applications. However, the system can also be incorporated into bench-top solid-state pH meters simplifying the calibration of a pH probe to a single aqueous solution measurement. The method can be used for any solid-state pH sensor, including ISFET based sensors.

### BRIEF DESCRIPTION OF THE DRAWINGS

Embodiments of the present invention will now be described by way of example only with reference to the accompanying drawings where like parts are provided with corresponding reference numerals and in which:

FIG. 1 schematically illustrates a pH sensor according to an embodiment of the invention;

FIG. 2 schematically illustrates a side view of a combined pH and DO sensor;

FIG. 3 schematically illustrates a top-down view of the sensor of FIG. 2;

FIG. 4 schematically illustrates a combined pH and DO sensor according to another embodiment of the invention;

FIG. 5 schematically illustrates an alternative electrode structure according to another embodiment;

FIG. 6 schematically illustrates another electrode structure according to a further embodiment;

FIG. 7 schematically illustrates a change in pH over time at a pH sensing electrode when a current is applied between a pair of electrolysis electrodes;

FIG. 8 is a schematic flow diagram illustrating a pH and DO sensor calibration method;

FIG. 9 is a schematic flow diagram illustrating a first pH calibration method; and

FIG. 10 is a schematic flow diagram illustrating a second pH calibration method.

### DETAILED DESCRIPTION

The present embodiment of the invention provides a smart sensor which is capable of determining pH and DO content within a measurement environment, such as a uterus or other organ of a human or animal body. Such a smart sensor may also be used in other remote environments, such as long-term environmental monitoring (for example in the ocean). The high-level structure and operation of each of a solid-state pH sensor and DO sensor suitable for use with the present invention will now be described:

### pH Sensor

A solid-state pH sensor comprises a pH sensitive electrode and a reference electrode provided within an electrolyte. Different kinds of micro-scale solid state pH sensors have been developed, including a metal oxide-based pH sensor, which has good pH measurement capabilities, a simple structure and can be micro-fabricated. A wide variety of metal oxides have been characterised and iridium oxide (IrOx) is commonly used. A typical IrOx pH sensor is made from a thin IrOx film (IROF) deposited onto an electrode. A simple structure enables simple fabrication, the use of a relatively simple processing circuit and small size. Such a pH sensor includes a pH sensing electrode (formed of IrOx) and a reference electrode, formed of Ag/AgCl. The electrode potential of the pH sensing electrode has a linear relationship with the pH of an ambient solution, according to the Nernst-equation, as will be discussed below. The open circuit potential (OCP) (versus the reference electrode) is used to measure the electrode potential and the pH value can be derived in accordance with the known relationship/expression. Given the linear response of the electrode potential with respect to pH, the calibration of such a pH sensor involves determining a fixed offset and the slope of the expression. In practice, it is the offset which varies most over time and is most important for correction.

The ideal relationship between electrode potential E and pH for pH electrodes is shown in the Nernst equation 1.

$$E = {E}^{0} - 2.3 \frac{R T}{F} pH \qquad ( Equ 1 )$$

E₀ represents the formal potential (fixed offset), defined by the reference electrode. Over recent decades, numerous materials have been investigated for use in pH sensing applications. One material of particular interest is Iridium Oxide (IrOx). This, in fact, is a metal oxide-metal oxide based system, having a response (variation in electrode potential as a function of pH) which is determined by the reaction occurring between its oxidation states. The IrOx pH sensor holds the following advantages: a linear response, low temperature coefficient, applicable in harsh environments, low impedance. Additionally, its simplicity of fabrication and biocompatibility make it a promising candidate for in vivo biomedical studies. The working principle of these pH sensors relies on the formation of insoluble hydroxide groups on the metal oxide surface when placed in solution. This formation allows for proton displacement to occur between the hydroxyl sites in which protons from the electrode surface are exchanged with the bulk solution, resulting in an electron transfer reaction. In simple terms, the interaction between the surface of the metal oxide and the pH of the solution causes a change in surface potential. Ostensibly, the equilibrium reaction, causing the pH sensitivity for an Iridium-oxide based sensor, is reliant on the electron exchange reaction shown in equation 2, and involves the Ir³⁺ and Ir⁴⁺ oxidation states.

2IrO₂+2H⁺+2e⁻↔Ir₂O₃+H₂O (Equ 2)

It will be understood that Ir₂O₃ represents an Ir⁺ oxidation state and that IrO₂ represents an Ir⁴⁺ oxidation state. The corresponding Nernst-equation is shown in equation 3, noting that pH=−log(H⁺):

$$E = {E}^{0} + \frac{R T}{nF} ln ( \frac{[ {Ir}^{4 +} ] [ {H}^{+} ]}{[ {Ir}^{3 +} ]} ) = {E}^{{0}^{′}} - 2.303 \frac{R T}{F} pH$$

$$were {E}^{{0}^{′}} = {E}^{0} + \frac{2.3 \; 03 RT}{nF} log ( \frac{[ {Ir}^{4 +} ]}{[ {Ir}^{3 +} ]} )$$

In equation 3, E is the electrode potential of the pH sensitive electrode, E⁰ is the reference electrode potential. E⁰′ is the complete formal potential, which can be seen to be a function of both the reference electrode potential E⁰, and the ratio of activity of the Ir⁴⁺ and Ir³⁺ states. R is the gas constant, T is temperature in Kelvin, n is the number of electrons and F is Faraday's constant. H⁺ is the activity of the hydrogen ions in the solution. Activity (of the H⁺, Ir³⁺ and Ir⁴⁺ states) is a measure of the effective concentration of the respective species under non-ideal (e.g. concentrated) conditions, as would be understood by a person skilled in the art. Reviewing the Nernst-equation 3, it can be seen that two factors would influence the stability of the pH sensor: sensitivity factor (the rate of change of electrode potential as a function of pH change, that is, the slope of the equation) and formal potential. For a long term implantable system, the potential needs to remain stable at fixed pH. The resulting potential from the IrOx sensor can be rewritten in terms of the activity of its two active oxides and the proton activity, as shown in equation 3. As can be seen, a redox system consisting of the Ir³⁺and Ir⁴⁺ is present. The amount of each species will determine the response of the fabricated sensor and is dependent on the method of deposition.

Most of the problems related to IrOx sensors are due to drift of the formal potential. In contrast, the sensitivity is less prone to change. When the formal potential drift has shifted too much by either a drifting reference electrode or a change in the IrOx ratio recalibration is required.

### DO Sensor

An electrochemical DO sensor uses the electrochemical reduction of dissolved oxygen (DO) at a microelectrode. The micro electrodes may be readily fabricated onto silicon substrates with micro-fabrication technologies and can be constructed out of different materials including platinum, gold and carbon. The most common electrochemical DO sensor is of the Clark type. Electrochemical DO sensors utilize a two or three electrode system, comprising a working electrode and a reference electrode/counter electrode (separate in a three electrode system). The working electrode is made from an inert metal, such as platinum or gold, and the electrochemical reactions of interest occur on its surface. The reference electrode is a nonpolarizable electrode, and has a stable and well-known electrode potential. For example, a widely used reference electrode for electrochemical purposes is Ag/AgCl electrode.

The electrochemical DO sensor is immersed in an electrolyte. When a negative voltage is applied to the working electrode against the reference electrode, dissolved oxygen in the electrolyte is consumed according to the following chemical reactions.

O₂+4e⁻+2H₂O↔4OH⁻ (Equ 4)

O₂+42+2H₂O↔H₂O₂+2OH⁻ (Equ 5)

H₂O₂+2e⁻↔2OH⁻ (Equ 6)

These reactions produce an electrical current through the working electrode which can be measured by an electronic circuit. In the case of a two-electrode system, this current flows through the working electrode and the reference electrode. In the case of a two-electrode system, this current flows through the working electrode and the counter electrode. Following the theory of oxygen reduction, upon sweeping the voltage between working electrode and reference electrode toward the potential of oxygen reduction an initial increase in current is observed. After a given period of time the current reaches a limit; its steady-state. This value is limited by the diffusion of oxygen to the working electrode surface. The magnitude of the observed current varies with dissolved oxygen content.

For low-power applications, prolonged operation of the DO circuitry is undesirable. Instead, a transiently operated DO measurement can be employed. Here, a rest voltage, defined as the voltage applied to the electrodes without any oxygen reduction resulting in zero current is applied prior to measurement. Next a measurement voltage, defined as the optimal voltage to achieve oxygen reduction is applied. By applying this step wave form, a transient current response is observed. When the measurement voltage is applied to the working electrode, the dissolved oxygen around the electrode is consumed. A current spike occurs at the beginning because of the fast potential change. Over time the current decreases rapidly due to oxygen consumption. At the same time, an oxygen concentration gradient is formed around the electrode. This oxygen concentration gradient causes oxygen molecules to diffuse to the working electrode from the bulk. Here, the oxygen reduction current approaches its steady-state value limited by oxygen diffusion. By taking fixed points on the transient profile a calibration curve can be obtained. Overall, the time taken for this type of measurement is in the millisecond range. Hence, the power consumption of the system is significantly reduced.

Over time, the reference electrode suffers from fouling and/or degradation. This results in a varying potential between working electrode and reference electrode shifting the optimal potential for both rest and measurement voltage.

Calibration of a dissolved oxygen sensor, in terms of setting the relationship between oxygen reduction current and dissolved oxygen concentration, may be achieved by operating the dissolved oxygen sensor in the presence of a known (and preferably high) dissolved oxygen concentration at the working electrode.

pH Sensor with Calibration Function

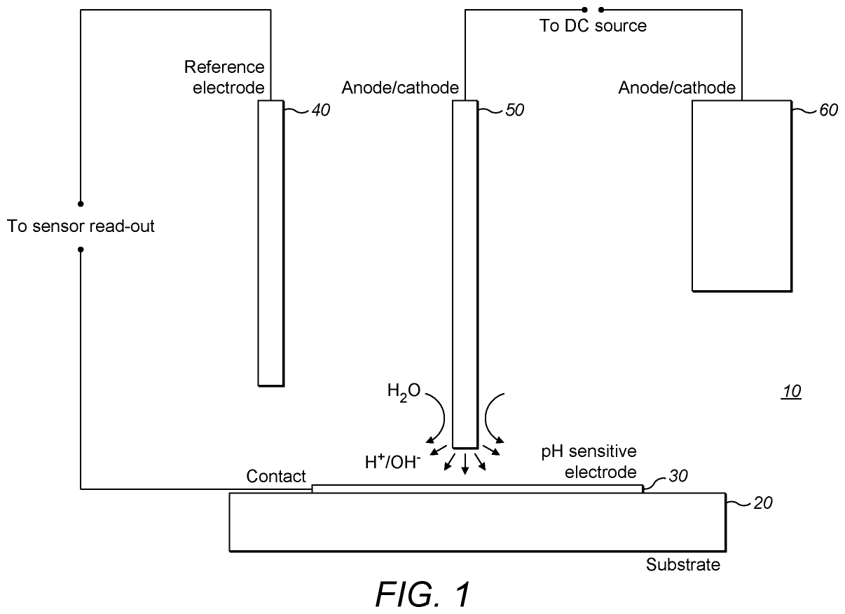

Referring to FIG. 1, a pH sensor 10 is shown. The sensor 10 comprises a substrate 20 on which is formed a pH sensitive electrode 30. The substrate 20 may be a glass substrate, which is impermeable to the electrolyte and measurement solution. A reference electrode 40 is provided, which may be formed of Ag/AgCl. The pH sensitive electrode 30 and the reference electrode 40 are connected to a sensor read-out (not shown) which measures a potential (voltage) difference between the pH sensitive electrode 30 and the reference electrode 40. The sensor read-out may be part of a controller (control circuitry) which serves to measure voltage and/or current levels across and through the various electrodes. As discussed above, the potential difference is related to the pH at the pH sensitive electrode. The sensor 10 also comprises an anode (or cathode) 50 and a cathode (or anode) 60, forming a pair of electrolysis electrodes, which are connected to a DC source (not shown) able to supply a constant current as part of a recalibration process. The anode (or cathode) 50 is proximate the pH sensitive electrode 30. By applying a DC potential difference (at constant current) between the anode (or cathode) 50 and the cathode (or anode) 60, an electrolysis reaction is initiated.

The electrolysis of water generates products according to the following reaction:

2H₂)↔O₂+4H++4e⁻ (anode) (Equ 7)

4e⁻+4H₂O↔2H₂+4OH⁻ (cathode) (Equ 8)

The above reactions at the anode and cathode (equations 7 and 8 respectively) can be initiated at electrode surfaces by applying a DC potential difference between the electrodes 50, 60 of at least 1.23V. By doing so, a local change in pH to either more acidic or more alkaline, dependent on the polarisation of the potential difference, is generated. If the distance between the pH sensing electrode 30 and generating electrode 50 is known, the change in pH at the pH sensing electrode 30 can be estimated based on the diffusion of H₃O⁺ and OH⁻. These estimations over time can then be used to obtain a calibration curve (curve in this context may include a straight line). From this, the new state of the sensor is determined and more accurate values of pH levels can be obtained.

More particularly, during calibration, a fixed voltage and constant current are applied across the electrodes 50, 60. The resulting electrolysis reaction will start to generate H⁺(H₃O⁺) or OH⁻ ions at the electrode 50, near the pH electrode 30, depending on the polarity of the applied voltage. This will cause the pH to either rise, or fall, again depending on polarity. The pH will therefore change over time, and the potential difference between the pH electrode 30 and the reference electrode 40 will therefore also change over time. The pH generated by the electrolysis reaction proximate the pH sensitive electrode at any given time during the calibration process is predetermined (that is, an expected value of pH given the amount of time over which the voltage has been applied across the electrodes 50, 60 is known). Accordingly, it is possible to map the voltage measured at the pH electrode 30 to a pH level known to be present at the electrode 30 at the time the voltage was measured. It will be appreciated that the magnitude of the pH changes caused by the electrolysis reaction will dominate over any fluctuations in pH present in the electrolyte itself prior to calibration, and so such fluctuations should not have a significant material effect on the calibration process.

This type of calibration can be performed either with the pH sensor 10 in direct contact with the measurement solution or through separation of the sensor by an internal (an)ion conductive electrolyte and membrane. In the latter case, the sensed pH change is dependent on the diffusion of the products within the internal electrolyte, preventing natural convection to take effect, and keeping out interfering species.

In FIG. 1, a pair of electrolysis electrodes are used in combination with the pH sensitive electrode and reference electrode of a solid-state pH sensor. The electrolysis electrodes are shown to be dedicated electrolysis electrodes (anode and cathode) separate from the pH electrode and the reference electrode. They are dedicated in the sense that they serve no purpose other than recalibration.

In one alternative implementation, the first electrode of the pair of electrolysis electrode is the pH sensitive electrode itself. The second electrode is separate from the pH sensitive electrode and the reference electrode. In this case, the pH sensor with calibration function can be achieved with three electrodes rather than four. However, as the pH measurement cannot be taken at the same time as a DC reference voltage is applied across the pH measurement electrode (acting as the first electrode) and the second electrode, the application of the DC reference voltage will need to be discontinued for a short time while the pH measurement is made. The discontinuation of the DC reference voltage (and thus the taking of the pH measurement) would take place after a predetermined duration of application of the DC reference voltage—at which time the pH level at the pH measurement electrode could be expected to be a first known value. Once the pH measurement has been taken, the DC reference voltage can be applied again for a further predetermined duration before another pH measurement is taken (at a time at which the pH level at the pH measurement electrode could be expected to be a second known value). This process can be continued until sufficient pH data points have been obtained to enable an accurate recalibration.

In another alternative implementation, the first electrode of the pair of electrolysis electrodes is the pH sensitive electrode itself, and the second electrode of the pair of electrolysis electrodes is the reference electrode. In this case, the pH sensor with calibration function can be achieved with two electrodes rather than three or four, but the issues associated with the three-electrode implementation also arise, and in addition this may place constraints on the type of reference electrode which can be used. In particular, this implementation is only viable if the reference electrode is capable of passing the current required to cause the electrolysis reaction. An Ag/AgCl reference electrode would not survive this, but a platinum electrode for example can pass it but is in other senses less effective as a reference electrode. Other reference electrodes can also be used such as graphene/carbon.

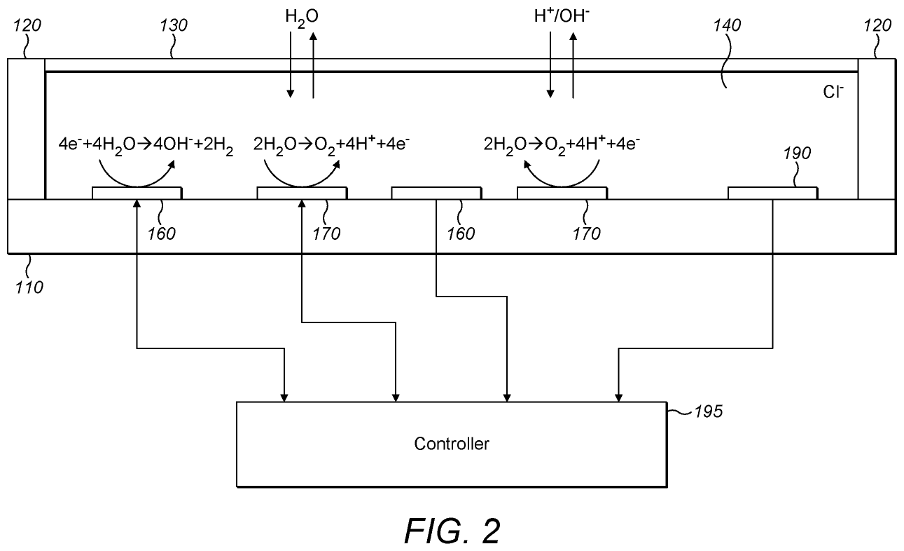

FIG. 2 extends the implementation of FIG. 1 by recognising that the pair of electrolysis electrodes could be implemented as the working electrode and counter electrode of a three-electrode dissolved oxygen sensor, thus permitting a combined pH and DO sensor to be provided with pH calibration (and also, as will be discussed below, DO calibration) without adding any further electrodes beyond those already required for pH and DO sensing (or pH sensing with calibration).

Referring to FIG. 2, a cross sectional view though a combined sensor 100 shows a substrate 110 upon which the various electrodes of the sensor are formed, side walls 120 and a semi-permeable membrane 130. The substrate 110, side walls 120 and membrane 130 define a substantially sealed unit which can be placed into a solution/sample which is to be measured. Within the sealed unit an electrolyte solution 140, in this case Chloride Cl⁻, is provided. The electrolyte solution 140 may be a gel, or liquid. The electrolyte is therefore separated from the measurement solution by the membrane 130, which permits transmission of ions (H⁺/OH⁻) and other chemicals (for example dissolved oxygen, in the form H₂O) via diffusion through the membrane 130. As discussed above, dissolved oxygen (for example) diffuses across the membrane 130 at a rate proportional to the pressure of oxygen within the measurement solution. Similarly, the pH within the measurement solution will cause pH changes within the electrolyte by diffusion of ions through the membrane 130.

Upon the substrate, several electrodes are provided. In particular, a pH sensitive electrode 160, a first generating electrode 170, a second generating electrode 180 and a reference electrode 190. The pH sensitive electrode 160 corresponds to the pH sensitive electrode 30 of FIG. 1. The reference electrode 190 corresponds to the reference electrode 40 of FIG. 1, and also serves as a reference electrode of the DO sensing part of the combined sensor 100. The first generating electrode 170 corresponds to the electrode 50 of FIG. 1, but also serves a working electrode of the DO sensing part of the combined sensor 100. The second generating electrode 180 corresponds to the electrode 60 of FIG. 1, but also serves as a counter electrode of the DO sensing part of the combined sensor 100. The pH sensitive electrode 160, the first generating electrode 170, the second generating electrode 180 and the reference electrode 190 are all electrically connected to a controller 195. The controller 195 comprises circuitry for measuring the potential difference across the pH sensitive electrode 160 and the reference electrode 190 during a pH measurement process. The controller 195 also comprises circuitry for applying a potential difference across the reference electrode 190 and the first generating electrode 170 and measuring a resulting current flow through the first generating electrode 170 (and thus the second generating electrode 180) in order to measure the dissolved oxygen concentration at the first generating electrode 170. The controller 195 also comprises circuitry for applying a DC recalibration voltage across the first generating electrode 170 and the second generating electrode 180 in order to modify the pH in the vicinity of the pH sensitive electrode 160, and as will be discussed subsequently to increase the dissolved oxygen concentration at the first generating electrode 170. The electrical circuitry within the controller 195 required to implement the above would be well known and understood by the skilled person. During measurement operations, a potential difference between the pH sensitive electrode 160 and the reference electrode 190 are used to determine the pH of the electrolyte 140, and thereby the pH of the measurement solution outside the sensor. Similarly, during measurement operations, a small first voltage (insufficient to cause electrolysis) is applied between the first generating (working) electrode 170 and the second generating (counter) electrode 180, and a resulting current proportional to the dissolved oxygen level in the electrolyte is measured and used to identify the dissolved oxygen level.

During pH calibration, a second voltage, greater than the first voltage, is applied between the first generating electrode 170 and the second generating electrode 180, causing the above-discussed electrolysis reactions to occur. In particular, it can be seen that the electrolysis reaction of equation 7 takes place at the first generating electrode 170 and the electrolysis reaction of equation 8 takes place at the second generating electrode 180. This results in the local pH at the first generating electrode 170 reducing (becoming more acidic), since the first generating electrode 170 is in this case the anode. The opposite change in pH can be achieved by reversing the polarity of the voltage applied between the first and second generating electrodes 170, 180. The first generating electrode 170 is proximate to the pH sensitive electrode 160, and so local changes in pH at the first generating electrode 170 will be experienced at the pH sensitive electrode. The second voltage is applied as a fixed voltage, and with a constant current. The potential difference between the pH sensitive electrode 160 and the reference electrode 190 (which is relatively distant from both the first generating electrode 170 and the second generating electrode 180 and will therefore not experience the effects of the electrolysis reactions) is periodically or continuously measured over time, and used to calibrate the pH sensor in the manner described above.

When the first generating electrode 170 is used as the anode, the electrolysis reaction (equation 7) which takes place generates oxygen as a by-product. As discussed previously, calibration of a dissolved oxygen sensor can be achieved by taking measurements at a high DO concentration. Accordingly, when the pH calibration has been complete, the oxygen concentration at the first generating electrode 170 can be expected to be high. Accordingly, by discontinuing the application of the second (higher) voltage at a constant current across the first and second generating electrodes 170, 180 and instead applying the first (lower) voltage across the first and second generating electrodes 170, 180 and measuring the resulting current, a calibration measurement at a high oxygen concentration can be obtained. In other words, the electrode structure and configuration of FIG. 2 permits (a) pH measurement, (b) DO measurement, (c) pH calibration and (d) DO calibration without the need for additional components, and as part of an integrated sensing and calibration procedure. The method described here can be used in a single on-chip electrode system when the pH sensor is used in combination with a dissolved oxygen (DO) sensor.

To summarise, the sensor, consisting of a platinum or gold working electrode, counter electrode and a reference electrode, can be configured electronically to form the anode and cathode to facilitate the generation of ions to calibrate the pH sensor. In this manner, the pH change is generated at the surface of the dissolved oxygen sensor. By adding the pH sensor in close proximity, the current status of the pH sensor is determined. In particular, a zero-crossing point (E₀′ in equation 3) and a slope (remainder of expression in equation 3) can be determined by determining a line of best fit to the sampled voltages. Additionally, the anodic reaction generates oxygen as a product of the electrolysis. By performing a transient dissolved oxygen measurement shortly after generating of H₃O⁺ and O₂ a single point calibration at a high DO concentration value can be obtained and used to assess the state of the DO sensor. This smart-sensor is able to measure both pH and DO accurately on a single system without the need for additional electrodes outside of the sensor package.

It will be understood that the electrolysis reaction could be carried out any number of times in any direction of polarisation, as to increase the pH from its base level and measure the resulting voltage changes, and to decrease the pH from its base level and measure the resulting voltage changes. In practice, since the relationship between voltage and pH is linear, this may not be required to identify the slope and zero crossing point of the sensor. Furthermore, in order to change the polarity of the applied voltage, a switching circuit will be required, thereby increasing complexity and size. It is preferable for the anode (rather than the cathode) of the electrolysis electrodes to be proximate/adjacent the pH sensitive electrode, since it is only the electrolysis reaction which takes place at the anode which generates the oxygen which also permits calibration of the DO sensor.

It will be appreciated that the calibration process artificially raises DO and pH levels, and that actual measurements of DO and pH levels of the measurement environment cannot be made until the levels in the electrolyte solution have returned to normal.

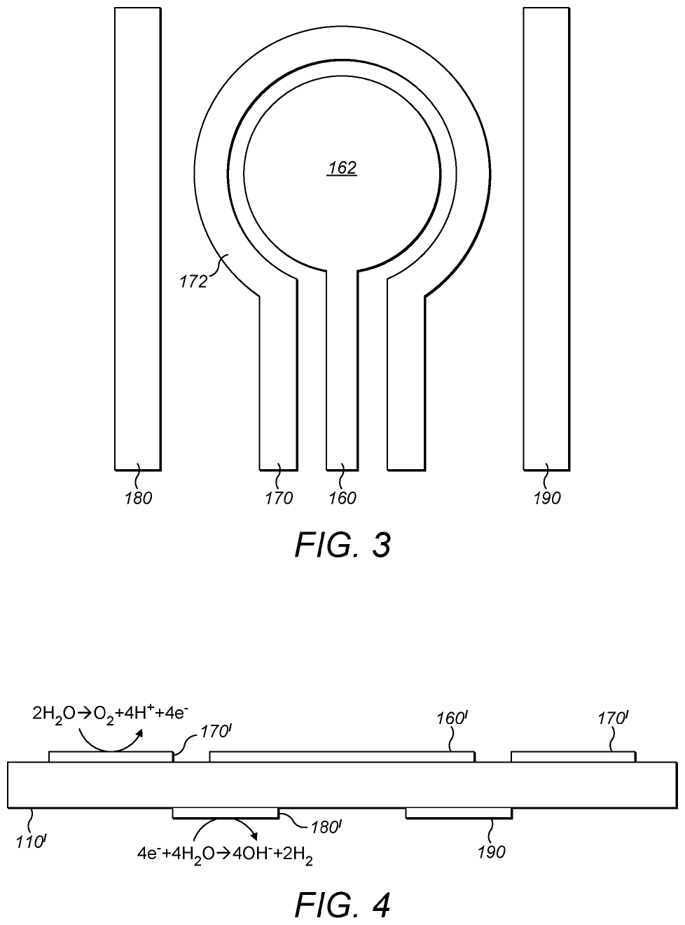

Referring to FIG. 3, this shows a top view of the electrode structure of FIG. 2. Like reference numerals are used to identify like components. It can be seen that the pH sensitive electrode 160 comprises a disk-like area 162, while the first generating electrode 170 comprises a ring area 172 which substantially surrounds the disk-like area 162 of the pH sensitive electrode 160. This places the disk-like area 162 and the ring area 172 in close proximity to strongly influence the ion and oxygen concentration at the pH sensitive electrode 160 during electrolysis. It will be appreciated that this arrangement could be reversed such that the pH sensitive electrode surrounds the first generating electrode. The distance between the pH sensitive electrode and the first generating electrode influences the time that would be required for the measurement to reach a desired pH value. A separation of 50 to 100 μm has been found to obtain a suitable pH shift within seconds. The time taken is also dependent on the current passed through the electrolysis electrodes. In particular, the higher the (constant) current which is applied, the faster the desired pH shift is achieved.

Referring to FIG. 4, this shows an alternative structure in which a pH sensitive electrode 160′ and a first generating electrode 170′ are provided on an upper side of the substrate 110, while a second generating electrode 180′ and a reference electrode 190′ are provided on a lower (opposite) side of the substrate 110. This has the added benefit that the OH⁻ generated on the cathode does not interfere with the generated H₃O⁺ which would otherwise cancel out the pH change if the distance between the two is too small.

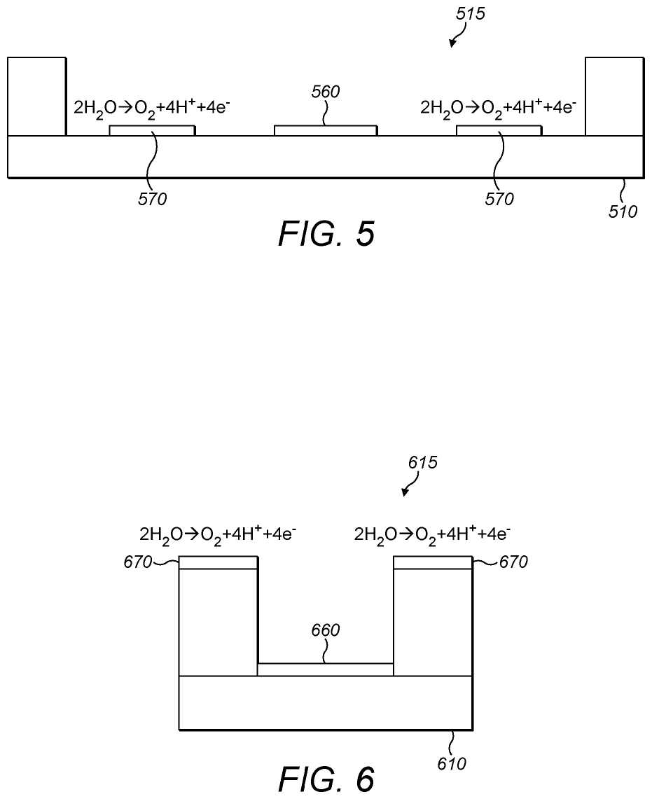

Referring to FIG. 5, an alternative embodiment is schematically illustrated in which a substrate 510 comprises a recessed well 515 within which a pH electrode 560 and first generating electrode 570 are formed (on the base of the well). As the recalibration voltage is applied between the first and second generating electrodes, a pH change at the first generating electrode 570 will fill the well 515 allowing the solution within the proximity of the pH sensor 560 to reach a particular (known) pH value more quickly than with the above-described embodiments. Typically, the depth of the well 515 will be in the micro-meter range. The reference electrode and second generating electrode (neither of which are shown in FIG. 5) are placed outside of and preferably away from the well. For example, the reference electrode and second generating electrode may be provided to the other side of the substrate 510 in a similar manner to FIG. 4. The shape of the well 515 when viewed from above (in plan view) preferably substantially follows the perimeter of the first generating electrode 570, to be substantially circular in the present example. The well 515 may be formed as a ridge extending substantially around the pH electrode 560 and first generating electrode 570. In FIG. 5, the pH electrode 560 is substantially surrounded by the first generating electrode 570 in like manner to FIG. 3. However, the first generating electrode 570 and the pH sensor 560 can be reversed, such that the pH electrode surrounds the first generating electrode.

Referring to FIG. 6, another alternative embodiment is schematically illustrated in which a substrate 610 comprises a recessed well 615 within which a pH electrode 660 is formed (on, and substantially covering, the base of the well 615). As with FIG. 5, the well 615 may be formed as a ridge extending substantially around (in this case) the pH electrode 660. A first generating electrode 670 is formed on top of the ridge of the well 615. In this case, a pH change in the solution at the first generating electrode 670 will progress from the corners of the first generating electrode 670 into the well 615. As with FIG. 5, the second generating electrode and the reference electrode are disposed outside of and preferably away from the well, for example to the other side of the substrate 510 in a similar manner to FIG. 4. As with FIG. 5, the structure of FIG. 6 can be expected to allow the pH level in the vicinity of the pH electrode 660 to change more quickly due to the shelter provided by the well 615.

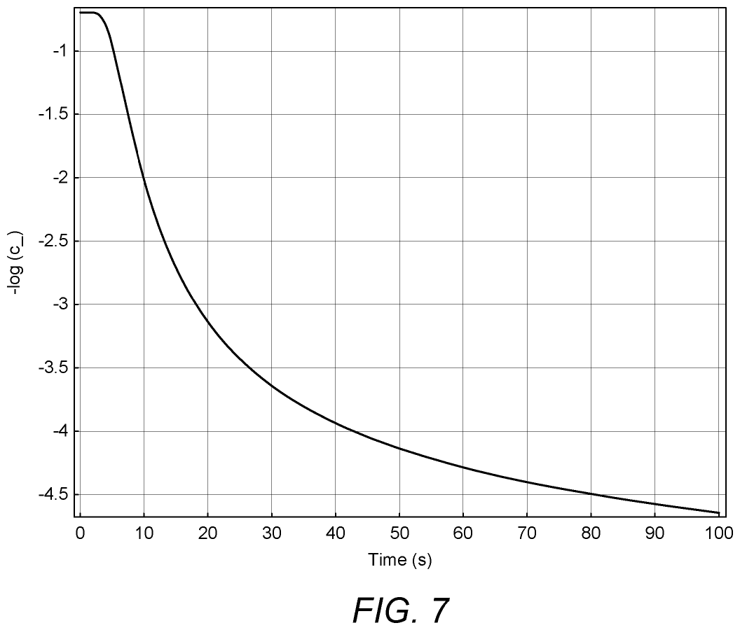

Referring to FIG. 7, an example change in pH (y axis) with respect to time (x axis) is illustrated when a constant current of 200 μA is applied across the first and second generating electrodes 170, 180 (at a fixed voltage of 1.23V and 50 uA cm−2 current density). It can be seen that the pH rapidly reduces during the first few seconds but then the rate of reduction slows due to the diffusion rate of water. It will be appreciated that the voltage measurements taken at various times during the electrolysis reaction can be mapped to the expected pH levels at those times (represented illustratively by the graph of FIG. 7) to calibrate the pH sensor.

The method for recalibrating the pH sensor may use either a two or three electrode electrolysis set-up. With a two-electrode set up, only the working electrode 170 and the counter electrode 180 are used, in the manner described above, but applying a constant voltage and current across them. The electrolysis electrodes 170, 180 in this case are ungrounded, which means that the absolute voltage at the first generating/working electrode 170 is unknown. With a three-electrode set up, the reference electrode 190 is used as a ground. In particular, an absolute voltage at the working electrode 170 is achieved by grounding the counter electrode 180 to the reference electrode 190. The three-electrode set up is particularly useful for variable environments, which with a two-electrode setup might result in an unpredictable absolute voltage being applied at the working electrode.

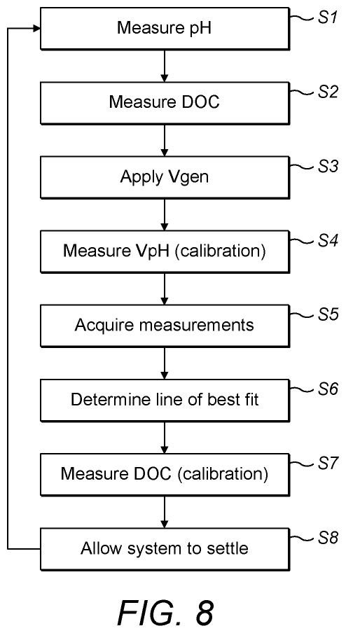

Referring to FIG. 8, a high-level flow diagram is provided to explain the overall operation of a combined pH and DO sensor with built in calibration. At a step S1, the potential V_{PH} at the pH sensitive electrode 160 with respect to the reference electrode 190 potential is measured over time. The measured potential is related to the current pH value of the electrolyte solution. At a step S2, a dissolved oxygen concentration is measured by applying a small fixed voltage across the first and second generating electrodes 170, 180 and measuring the current. The stable current is related to the DO concentration in the electrolyte solution. The steps S1 and S2 can be carried out in parallel, or can be interleaved. At a step S3, a calibration process is triggered, and in particular a higher fixed voltage (V_{gen}) at a constant current is applied between the first and second generating electrodes 170, 180. A local pH change is thereby generated over time at the surfaces of the first generating electrode 170 and the second generating electrode 180. As time progresses, the boundary of the pH change in the electrolyte solution starts to cover the pH sensor electrode 160 resulting in a change in V_{pH}. At a step S4, the voltage V_{pH} is repeatedly sampled over time. At a step S5, at least two points of V_{pH} at registered times are acquired, and are set to correspond with simulated or calculated values of the generated pH change across the pH sensor surface (that is, for example, the graph of FIG. 7). At a step S6, a linear fit is performed on the acquired points to obtain a new calibration curve/relationship between voltage and pH. At a step S7, the voltage V_{gen} is switched off, and a smaller voltage applied in order to measure the current through the working electrode 170 and counter electrode 180 at the high oxygen concentration resulting from the electrolysis reaction at the anode (first generating electrode 170). This serves to calibrate the DO sensor. At a step S8, the system is allowed to re-equilibrate with the measurement solution. Measurement at the steps S1 and S2 can then continue.

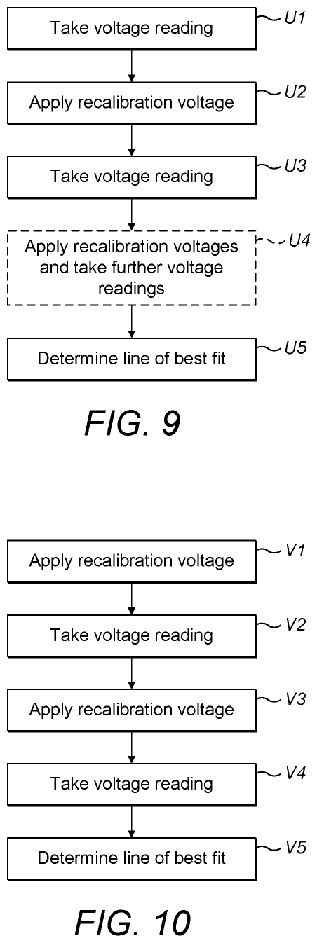

Referring to FIGS. 9 and 10, two possible pH calibration methods using the present technique are described. The method of FIG. 9 is effective where the pH of the environment in which the pH sensor is disposed at the time of calibration is known, or is unknown but constant. The method of FIG. 10 is effective both under the same circumstances as the method of FIG. 9, but is also effective where the pH of the environment in which the pH sensor is disposed at the time of calibration is neither known, nor can be assumed to be constant.

In FIG. 9, at a step U1, a voltage reading is taken at a known/initial pH, providing a first calibration point. This reading is taken before a voltage is applied to the pair of electrolysis electrodes—that is, before the local pH is modified by electrolysis. At a step U2, a recalibration voltage/current is applied to the pair of electrolysis electrodes for a predetermined period of time. As the electrode geometry (that is, the shape and separation between the pH sensitive electrode and the first electrolysis electrode), the voltage, the current and the duration of its application are all known and predetermined, a pH change (increase or decrease) associated with the application of the recalibration voltage/current is also known. The pH change can be added or subtracted from the previous pH value (depending whether the change is a reduction of pH or an increase in pH) to give a new actual pH value. At a step U3, a voltage reading is taken at the end of the predetermined period of time and is associated with the known pH at that time, to provide a second calibration point. Optionally, at a step U4, one or more further applications of the recalibration voltage are applied. These may be of the same polarity as the voltage applied in the step U2 to extend the pH change further in the same direction, or of an opposite polarity to obtain a further calibration point in the opposite direction. It will be appreciated that any desired number of recalibration points may be obtained in this way, at various points on the pH scale. At a step U5, a linear fit is performed on the recalibration points to obtain both the sensitivity and formal potential of the Nernst equation. These will then be used during future operation of the pH sensor, in mapping a read voltage to a particular pH value.

In FIG. 10, at a step V1, a recalibration voltage/current is applied to the pair of electrolysis electrodes for a predetermined period of time (which may be different to the predetermined period of FIG. 9) known to result in a first steady-state pH at the pH sensitive electrode. The recalibration voltage is then switched off. Again, the period of time required to achieve this, and the pH value reached, is known because the electrode geometry, the applied voltage and current are all known and predetermined. At a step V2, a voltage reading is taken while the pH is at the first steady state, and is associated with the known, steady state, pH value, to provide a first calibration point. At a step V3, the recalibration voltage is applied again, but with the polarity being reversed. Here, the recalibration voltage is applied for a further predetermined period of time (which may be the same as that of the step V1 if the pH level at the pH sensitive electrode has already settled, or may be of greater duration if the pH change arising from the step V1 first needs to be reversed), again, until a steady state pH is achieved. At a step V4, a voltage reading is taken while the pH is at the second steady state, and is associated with the known, steady state, pH value, to provide a second calibration point. At a step V5, a linear fit is performed on the first and second recalibration points to obtain both the sensitivity and formal potential of the Nernst equation. These will then be used during future operation of the pH sensor, in mapping a read voltage to a particular pH value.

It will be understood that the techniques and structures described herein could be applied to intra-uterine monitoring (by implementing a combined pH and DO sensor in an implantable sensor device), to other body-cavity monitoring, such as within a vagina, bladder or digestive tract of a human or animal body. Furthermore, the pH sensor and calibration method (optionally with the DO sensor) could be used in other applications such as remote environmental modelling or laboratory instrumentations. The present technique is relevant in any application in which long term remote monitoring of a pH level is required, since the long term use requires recalibration to compensate for (for example) drift, while the remote use means that such recalibration must be carried out in situ rather than by removing the sensor from the measurement environment. In addition to the structures described above, a sensor device containing a pH sensor and optionally a DO sensor can be expected to comprise components such as an antenna and transmitter/receiver circuitry for communicating sensor readings to an external device, and optionally for receiving control signals for controlling the sensor device. Such transmitter/receiver circuitry would be interfaced with, or part of, the controller 195 described above, or equivalent controllers applied to the other embodiments of the invention described herein.

The present invention is not limited to a specific pH sensitive material. Suitable materials could include (but are not limited to) different metal oxides, ISFETS, or hydrogels. Further, various fabrication aspects of the system such as the separation distance between electrodes and electrode materials can be varied in dependence on the application, requirements and materials used. Moreover, various different types of electrode lay-out can be used, for example disks, rings and interdigitated electrodes.

It will be appreciated that the calibration could take place on-chip (that is, within the sensor itself), or alternatively the sensor may simply apply voltages and measure voltages and currents for transmission externally of the sensor, with the calibration being applied to the voltage and current measurements being output by the sensor by a device in receipt of such voltage and/or current measurements. Similarly, the sensor may itself fully control the calibration process of triggering electrolysis, or may alternatively be responsive to a received instruction from outside the sensor to trigger the electrolysis reaction.

1. A pH sensor for measuring pH levels within a measurement environment, the sensor comprising:
a reference electrode;
a pH sensitive electrode,
a controller, for measuring the potential difference between the pH sensitive electrode and the reference electrode, the measured potential difference being indicative of a pH level at the pH sensitive electrode;
and
wherein the controller is operable:
to apply a voltage across first and second electrodes to control the pH level at the pH sensitive electrode, and
to measure the potential difference between the pH sensitive electrode and the reference electrode following the application of the voltage.

2. A pH sensor according to claim 1, wherein the first electrode is the pH sensitive electrode.

3. A pH sensor according to claim 1, wherein the second electrode is separate from the pH sensitive electrode and the reference electrode.

4. A pH sensor according to claim 1, wherein the second electrode is the reference electrode.

5. A pH sensor according to claim 1, wherein the first and second electrodes are separate from the pH sensitive electrode and the reference electrode, the first electrode being disposed in the vicinity of the pH sensitive electrode.

6. A pH sensor according to claim 1, wherein the controller is operable to recalibrate the pH sensor based on the measurement.

7. A pH sensor according to claim 1, wherein the controller is operable, while the voltage is being applied, to take a plurality of potential difference measurements at known times following the initial application of the voltage, and is operable subsequently to correlate the plurality of potential difference measurements with expected pH levels at the respective known times.

8. A pH sensor according to claim 1, wherein the controller is operable to apply the voltage with a first polarity until a first steady-state pH level is reached, and to take a first potential difference measurement at the first steady-state pH level, and then to apply the voltage with a second polarity opposite to the first polarity until a second steady-state pH level is reached, and to take a second potential difference measurement at the second steady-state pH level.

9. A pH sensor according to claim 7, wherein the controller is operable to determine a line of best fit through the correlated potential difference measurements and pH levels to obtain a gradient and/or zero crossing for a function used to determine pH level from measured voltage.

10. A pH sensor according to claim 1, wherein the first electrode substantially surrounds the pH sensitive electrode.

11. A pH sensor according to claim 10, wherein the pH sensitive electrode comprises a substantially disk-shaped part and the first electrode comprises a ring-shaped part which substantially surrounds the disk-shaped part of the pH sensitive electrode.

12. A pH sensor according to claim 1, wherein the pH sensitive electrode and the first electrode are formed on a substrate within a well.

13. A pH sensor according to claim 1, wherein one of the pH sensor and the first electrode are formed on a substrate within a well and the other of the pH sensor and the first electrode are formed on a raised area surrounding the well.

14. A pH sensor according to claim 12, wherein the reference electrode and the second electrode are formed outside of the well.

15. A pH sensor according to claim 1, wherein the pH sensor is an ISFET or metal oxide based sensor.

16. A pH sensor according to claim 1, wherein the measurement environment is within a human or animal body.

17. A pH sensor according to claim 16, wherein the measurement environment is within a uterus.

18. A multi-sensor device comprising the pH sensor according to any preceding claim and a dissolved oxygen sensor, the dissolved oxygen sensor comprising the first and second electrodes, one of the first and second electrodes being used as the working electrode of the dissolved oxygen sensor.

19. A multi-sensor device according to claim 18, wherein the other of the first and second electrodes is used as a counter electrode of the dissolved oxygen sensor.

20. A multi-sensor device according to claim 18, wherein the reference electrode of the pH sensor is a common reference electrode for use with the dissolved oxygen sensor.

21. A multi-sensor device according to claim 18, wherein the controller is operable to measure a dissolved oxygen level at the working electrode following the application of the voltage to obtain a single point calibration of the dissolved oxygen sensor at a high dissolved oxygen concentration.

22. A multi-sensor device according to claim 18, wherein the pH sensitive electrode, the reference electrode and the first and second electrodes are disposed on a substrate.

23. A multi-sensor device according to claim 22, wherein the substrate is a glass substrate.

24. A multi-sensor device according to claim 22, wherein the pH sensitive electrode, the reference electrode and the first and second electrodes are disposed on the same side of the substrate.

25. A multi-sensor device according to claim 22, wherein the pH sensitive electrode and the first electrode are disposed on a first side of the substrate and the reference electrode and the second electrode are disposed on a second, opposite, side of the substrate.

26. A multi-sensor device according to claim 22, wherein an electrolyte is provided in a region bounded by the substrate and a semi-permeable membrane, the region containing at least the pH sensitive electrode and the first electrode, diffusion of ions and oxygen from the measurement environment into the electrolyte occurring via the semi-permeable membrane.

27. A method of calibrating a pH sensor within a measurement environment, the method comprising the steps of applying a voltage across first and second electrodes to influence the pH level at a pH sensitive electrode, and measuring the potential difference between the pH sensitive electrode and a reference electrode following the application of the voltage.

28. A method according to claim 27 for calibrating both the pH sensor and a dissolved oxygen sensor integrated with the pH sensor, comprising a step of measuring a dissolved oxygen level at the first electrode following the application of the voltage to obtain a single point calibration of the dissolved oxygen sensor at a high dissolved oxygen concentration.
